(19) United States
(12) Patent Application Publication
(10) Pub. No.: US 2019/0111181 A1
(43) Pub. Date: Apr. 18, 2019

(54) METHODS FOR DENUCLEATION OF BIOLOGICAL TISSUE

(71) Applicants: Ghassan S. Kassab, La Jolla, CA (US); Xiao Lu, San Diego, CA (US)

(72) Inventors: Ghassan S. Kassab, La Jolla, CA (US); Xiao Lu, San Diego, CA (US)

(21) Appl. No.: 16/158,910

(22) Filed: Oct. 12, 2018

Related U.S. Application Data
(60) Provisional application No. 62/571,588, filed on Oct. 12, 2017.

(51) Int. Cl. A61L 27/36

(52) U.S. Cl. CPC A61L 27/3687

(57) ABSTRACT

Methods for denucleation of biological tissue. A method of denucleating biological tissue can include exposing a target tissue to at least one hyperosmotic solution and at least one hypoosmotic solution in an alternating fashion, and then applying a glutaraldehyde solution to the target tissue to fix the extracellular matrix and cytoskeleton of the target tissue.

### PRIORITY

The present application is related to, and claims the priority benefit of, U.S. Provisional Patent Application No. 62/571,568, filed Oct. 12, 2017, the contents of which are incorporated herein directly and by reference in their entirety.

### BACKGROUND

The present invention relates generally to a method of preparation of biological tissue for implantation.

There are two major schools of thought for preparation of biological tissue for tissue engineering applications. These applications include, but are not limited to valve leaflets, grafts, tissue patches, etc. In one approach, tissue is fixed in order to cross-link the extracellular matrix and component structures, rendering the tissue non-immunogenic. In another approach tissue is decellularized in order to remove cells thereby avoiding the immune response and tissue reaction.

However, the presence of cells is actually advantageous as the smoothness of the tissue surface renders tissue less thrombogenic and the glycocalyx has a favorable biochemistry. Hence, it would be ideal to retain the epithelial cells and surface of the tissue but remove the DNA and RNA content of the nuclei, which are the most inflammatory or immunogenic molecules. Therefore the inventor has developed a novel process that removes the content of the nuclei while retaining the epithelial cells and surface of the tissues.

### BRIEF SUMMARY

The present disclosure describes a method of denucleating biological tissue, such as swine pulmonary visceral pleura, while also retaining the surface cells, thereby rendering the tissue less thrombogenic and more biochemically favorable while also minimizing the immune and inflammatory response.

In one preferred embodiment a method of denucleating biological tissue comprises the steps of: exposing a target tissue to at least one hyperosmotic solution and at least one hypoosmotic solution in an alternating fashion; and then applying a glutaraldehyde solution to the target tissue to fix the extracellular matrix and cytoskeleton of the target tissue.

In another preferred embodiment a method for denucleating biological tissue comprises the steps of: exposing a target tissue to at least one hyperosmotic solution and at least one hypoosmotic solution in an alternating fashion; rinsing the target tissue to remove cytoplasmic components expelled by the application of the at least one hyperosmotic and at least one hypoosmotic solutions; applying a DNase and a RNase to degrade the remaining nucleic acid fragments; and applying a glutaraldehyde solution to the target tissue to fix the extracellular matrix and cytoskeleton of the target tissue. In a further embodiment the method includes the step of applying a protease inhibitor to prevent degradation of an extracellular matrix of the target tissue during exposure to the at least one hyperosmotic solution and the at least one hypoosmotic solution. The method may also comprise the step of applying an antibiotic to prevent bacterial growth in the target tissue during exposure to the at least one hyperosmotic solution and at least one hypoosmotic solution.

In another preferred embodiment a method for denucleating biological tissue comprises the steps of: exposing a target tissue to at least one hyperosmotic solution and at least one hypoosmotic solution in an alternating fashion; applying a protease inhibitor to prevent degradation of an extracellular matrix of the target tissue during exposure to the at least one hyperosmotic and at least one hypoosmotic solutions; applying an antibiotic to prevent bacterial growth in the target tissue during exposure to the at least one hyperosmotic and at least one hypoosmotic solutions; rinsing the target tissue to remove cytoplasmic components expelled by the application of hyperosmotic and hypoosmotic solutions; applying a DNase and a RNase to degrade remaining nucleic acid fragments; and applying a glutaraldehyde solution to the target tissue to fix the extracellular matrix and cytoskeleton of the target tissue.

The target tissue may be first exposed to a hypoosmotic solution followed by a hyperosmotic solution in an alternating fashion, or the target tissue may be first exposed to a hyperosmotic solution, followed by a hypoosmotic solution in an alternating fashion.

In an alternate embodiment the target tissue is exposed to at least one hyperosmotic solution and at least one hypoosmotic solution. In a further embodiment the at least one hyperosmotic solution is two or more hyperosmotic solutions. In another embodiment the at least one hypoosmotic solution is two or more hypoosmotic solutions.

The glutaraldehyde solution may be at least 0.25%. Alternatively, the glutaraldehyde solution may be no greater than 0.25%. The glutaraldehyde solution may also be applied to the target tissue for at least 24 hours.

In one exemplary embodiment the target tissue comprises swine pulmonary visceral pleura.

The protease inhibitor may be phenylmethylsulfonyl fluoride (PMSF), or any protease inhibitor known in the art or combination of such inhibitors.

The antibiotic may be penicillin, streptomycin, any antibiotic known in the art, or any combination of such antibiotics. The antibiotic may be applied during exposure of the target tissue to the at least one hyperosmotic solution and the at least one hypoosmotic solution.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows an en face image of swine pulmonary visceral pleura nuclei after being fixed by glutaraldehyde and Hoechst staining (panel A) and an en face image of swine pulmonary visceral pleura nuclei after being fixed by glutaraldehyde and SYTO® green-fluorescent staining (panel B), according to exemplary embodiments of the present disclosure; and

FIG. 2 shows a transverse section of swine pulmonary visceral pleura Hoechst 33342 (panel A) and a transverse section of swine pulmonary visceral pleura after decellularization stained with Hoechst 33342 (panel B), according to exemplary embodiments of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure is not limited to the exemplary tissue used to explain the method and can be generalized to many other tissues types including but not limited to pericardium, mesentery, pleural ligament, placenta, dura mater, and other such membrane-type tissue with an epithelial layer. Additionally, although certain types of tissue applications may be mentioned, the disclosure is not intended to limit the current invention to these types of applications.

In an exemplary embodiment of a method of the present disclosure, the method comprises the steps of using combination of hyperosmotic and hypoosmotic solutions to expel the nuclear content, such as the DNA and RNA, followed by enzymatic degradation of nuclear material. The de-nucleated tissue is then fixed with glutaraldehyde to cross-line the cytoskeleton of the cell. This combination retains both the benefits of cell surface and cross-linking.

In one exemplary embodiment swine pulmonary visceral pleura (herein after referred to as SPVP) is used as an example. Glutaraldehyde fixation is a general method for preparation of SPVP and greatly diminishes immunologic response when a prosthesis composited of heterologous SPVP is implanted. However, the risk of inflammation induced by DNA and RNA fragments in SPVP may result in complications for implantation. Exogenous and endogenous DNA fragments may provoke inflammation through the monocyte and lymphocyte Toll-like receptor 9 (TLR9) or cyclic guanosine monophosphate-adenosine monophosphate (cGAMP). Endogenous DNA fragments may be from fetal cells, cancer cells, etc. Exogenous DNA fragments may be from bacteria (dialysis), homogeneous or heterogeneous (tissue or organ transplantation), etc.

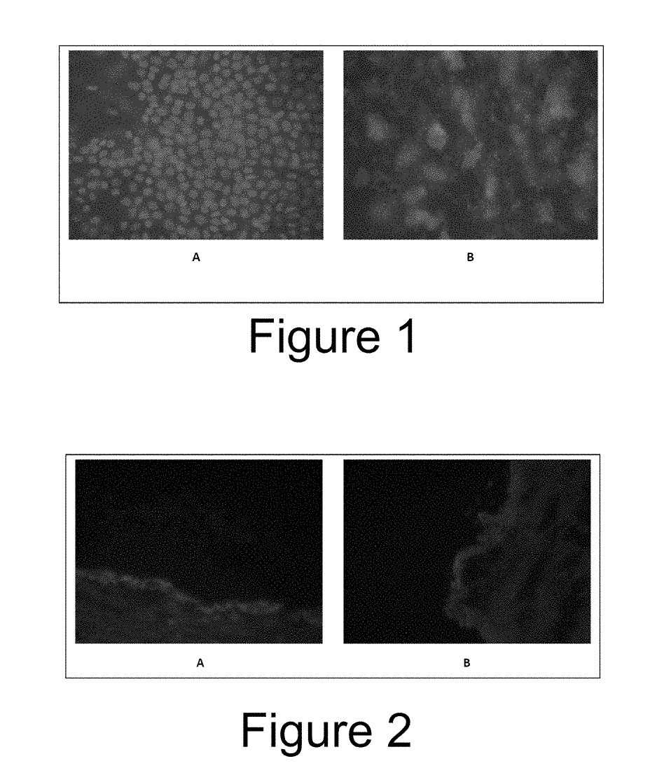

DNA and RNA fragments may remain in SPVP using glutaraldehyde fixation since glutaraldehyde does not crosslink DNA and RNA. Hopwood pointed “At temperatures up to 64° C. no reaction occurred between native DNA and glutaraldehyde. Reactions between RNA and glutaraldehyde were similar. There was little evidence for the formation of cross-links between nucleic acid molecules even at elevated temperatures (Hopwood D, Histochem J. 1975 May; 7(3):267-76.).” Accordingly, nuclei in SPVP after glutaraldehyde fixation could be stained using Hoechst 33342 (2′-[4-ethoxyphenyl]-5-[4-methyl-1-piperazinyl]-2,5′-bi-1H-benzimidazole trihydrochloride trihydrate) which binds preferentially to adenine-thymine regions of DNA and SYTO® green-fluorescent nucleic acid stains (ThermoFisher Scientific) which binds to nucleic acid, and underscores that DNA and RNA are rarely crosslinked by glutaraldehyde. FIG. 1 (panels A and B) show Hoechst staining and SYTO® green-fluorescent staining after SPVP is fixed by glutaraldehyde, respectively. In particular, FIG. 1 shows en face images of SPVP nuclei staining, with panel A showing Hoechst 33342 staining (objective 20×), and with panel b showing SYTO® green-fluorescent staining (objective 60×).

Decellularization is a procedure for removing cellular components (nucleic and cytosolic contents) in biologic tissue while retaining the bio-ability of the extracellular matrix. Decellularization diminishes the immunoreaction and inflammation from cellular components. For vascular and valve prostheses, the sole use of decellularization to prepare tissue may not be a preferred preparation since collagen, fibronectin, laminin, etc. in the extracellular matrix may elicit thrombosis. For the typical usage of biologic tissue in vascular prostheses, the procedures of denucleation and glutaraldehyde fixation can be combined to minimize the effects of inflammation from DNA and RNA fragments and thrombogenesis.

Generally, decellularization may be achieved through osmotic, detergent, and enzymatic methods. The osmotic method is used to alternatively expose biologic tissue in hypotonic and hypertonic solutions to expel cytosolic components such as proteins, DNA, RNA, etc. A detergent method uses detergents (e.g., Triton X-100, Sodium deoxycholate, etc.) to lyse cells and solubilize cellular and membrane components. An enzymatic method uses various enzymes to degrade proteins, DNA and RNA, e.g., trypsin thereby leaving the cell free from an extracellular matrix. This process is typically used in endothelial, mesothelial, and epithelial cells. Deoxyribonuclease (hereinafter referred to as DNase) and ribonuclease (hereinafter referred to as RNase) is used to degrade DNA and RNA. Antibiotics such as penicillin and streptomycin may be used to prevent bacteria growth during the process. Various protease inhibitors may also be used to prevent the degradation of extracellular matrix.

To prepare biologic tissue for vascular prostheses, an osmotic method is used to expel cytosolic components including nuclear matter like DNA and RNA. Cell membranes and nuclear envelopes are comprised of phospholipid bilayers whose permeability is highly selective. Small, nonpolar molecules move across phospholipid bilayers quickly. In contrast, large molecules and charged substances cross the phospholipid bilayers slowly. Therefore osmosis occurs when solutions are separated by the phospholipid bilayers' membrane that is permeable to some molecules but not to others. Hyperosmotic stress moves water out of the cells and nucleus and results in the cells and nucleus shrinking and the cell membrane and nuclear envelope shriveling. Hypoosmotic stress moves water into cells and the incoming water causes the cells and nucleus to swell or even burst. The osmotic method alternates hypoosmotic and hyperosmotic stresses to expel cytoplasma proteins and DNA and RNA out of cells.

A protease inhibitor, such as phenylmethylsulfonyl fluoride (PMSF), may be used to prevent possible degradation of extracellular matrix during the osmotic procedure. Intensive rinses are performed to remove the fragments of proteins, DNA and RNA. DNase and RNase are used to degrade residual DNA and RNA fragments.

The tissue is further fixed in low concentration glutaraldehyde (0.25%) for approximately 24 hours to crosslink antigens in cytoskeleton and extracellular matrix. The described process preserves the non-thrombogenic property of SPVP since the mesothelial cytoskeleton and the glycocalyx of SPVP are preserved from the osmotic method and the collagen, fibronectin, etc. are crosslinked by glutaraldehyde. This process reduces the probability of inflammation elicited by DNA and RNA fragments.

An exemplary pulmonary visceral pleura (PVP) decellularization protocol is as follows:

### 1. Harvest Tissue

After harvest, swine lung should be immediately stored in 4° C. saline for transportation. The PVP is gently peeled from swine lung with the aid of pressurized phosphate buffered saline (PBS) pumped into the interstitial space between the lung and PVP or pressurized air can be guided to the interstitial space between the lung and PVP.

### 2. First Osmotic Stress: Hypotonic Treatment

The tissue is placed in a Tris buffer of pH 8.0 and PMSF (10⁻⁶ M) for 24-48 hours. Optionally penicillin (100 U/ml) and streptomycin (100 U/ml) may be used.

### 3. Second Osmotic Stress: Hypertonic Treatment

The tissue is placed in a Tris buffer of pH 8.0 and KCl (1.5 M) and PMSF (10^{—6} M) for 24-48 hours. Optionally penicillin (100 U/ml) and streptomycin (100 U/ml) may be used.

### 4. Enzymatic Treatment for DNA/RNA Degradation

The tissue is placed in a Tris buffer of pH 8.0 and deoxyribonuclase (0.2 mg/ml) and ribonuclase (0.02 mg/ml) for 5 hours.

### 5. Rinse

The tissue is placed in a PBS Buffer of pH 7.4 for 72 hours.

### 6. Glutaraldehyde Crosslink

The tissue is placed in NaOH (2.05 g) and KH₂PO₄ (9.08 g) and deionized water (990 ml) and 25% glutaraldehyde (10 ml) for 24 hours.

### 7. Sterilization

The tissue is placed in 2.05 g/l NaOH, 10.83 g/l KH₂PO₄, 200 ml/l alcohol, 40 ml/l 25% glutaraldehyde, 110 ml/l 4% formaldehyde at 37° C. for 24 hours.

### 8. Rinse

The tissue is rinsed in sterilized saline 5 times for 40 min before implantation.

Immunofluorescence microscopy demonstrates that the mesothelial cytoskeleton and the glycocalyx were preserved from the osmotic method and DNase and RNase degradation. Panel A of FIG. 2 shows abundant heparan sulfate in the mesothelium in SPVP (paraformaldehyde-fixed). Panel B of FIG. 2 shows that heparan sulfate was still abundant in the mesothelium in denucleated SPVP (paraformaldehyde-fixed). Paraformaldehyde fixation was used for immunofluorescence since glutaraldehyde-fixation shields antigens. In particular, FIG. 2 shows immunofluorescence of heparin sulfate, with panel A showing a transverse section of SPVP, with red (portions of the upper part of the image) being heparin sulfate and blue (the remaining portions of the image, including the spotted portions) being nuclei staining with Hoechst 33342, and with panel B showing a transverse section of decellularized SPVP, with red (the portion indicated in the relative middle of the image) being heparin sulfate and with blue (the portion s indicated in the relative right side of the image) being nuclei staining with Hoechst 33342, noting that there is no nuclear material.

While various methods for denucleation of biological tissue have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method for denucleating biological tissue, the method comprising the steps of:
exposing a target tissue to at least one hyperosmotic solution and at least one hypoosmotic solution in an alternating fashion;
applying a glutaraldehyde solution to the target tissue to fix the extracellular matrix and cytoskeleton of the target tissue.

2. The method of claim 1, wherein the at least one hyperosmotic solution and at least one hypoosmotic solution is two or more hyperosmotic solutions and two or more hypoosmotic solutions, respectively.

3. The method of claim 2, wherein the glutaraldehyde solution is of at least 0.25%.

4. The method of claim 3, wherein the step of applying a glutaraldehyde solution to the target tissue to fix the extracellular matrix and cytoskeleton of the target tissue further comprises the step of:
applying the glutaraldehyde solution to the target tissue for at least 24 hours.

5. The method of claim 1, wherein the step of exposing a target tissue to at least one hyperosmotic solution and at least one hypoosmotic solution in an alternating fashion further comprises the step of:
exposing the target tissue to a hypoosmotic solution first, then exposing the target tissue to a hyperosmotic solution.

6. The method of claim 1, wherein the step of exposing a target tissue to at least one hyperosmotic solution and at least one hypoosmotic solution in an alternating fashion further comprises the step of;
exposing the target tissue to a hyperosmotic solution first, then exposing the target tissue to a hypoosmotic solution.

7. A method for denucleating biological tissue, the method comprising the steps of:
exposing a target tissue to at least one hyperosmotic solution and at least one hypoosmotic solution in an alternating fashion;
rinsing the target tissue to remove the cellular components expelled by the exposure to hyperosmotic and hypoosmotic solutions,
applying a DNase and a RNase to degrade remaining nucleic acid fragments; and
applying a glutaraldehyde solution to the target tissue to fix the extracellular matrix and cytoskeleton of the target tissue.

8. The method of claim 7, wherein the glutaraldehyde solution is no greater than 0.25%.

9. The method of claim 7, further comprising the step of:
applying a protease inhibitor to prevent degradation of a extracellular matrix of the target tissue during exposure to the at least one hyperosmotic and at least one hypoosmotic solutions.

10. The method of claim 7, further comprising the step of:
applying an antibiotic to prevent bacterial growth in the target tissue during exposure to the plurality of hyperosmotic and hypoosmotic solutions.

11. A method for denucleating biological tissue, the method comprising the steps of:
exposing a target tissue to at least one hyperosmotic solution and at least one hypoosmotic solution in an alternating fashion;
applying a protease inhibitor to prevent degradation of a extracellular matrix of the target tissue during exposure to at least one of the hyperosmotic or hypoosmotic solutions;
applying an antibiotic to prevent bacterial growth in the target tissue during exposure to at least one of the hyperosmotic or hypoosmotic solutions;
rinsing the target tissue to remove cellular components expelled by the exposure to hyperosmotic and hypoosmotic solutions,
applying a DNase and a RNase to degrade remaining nucleic acid fragments; and
applying a glutaraldehyde solution to the target tissue to fix the extracellular matrix and cytoskeleton of the target tissue.

12. The method of claim 11, wherein an antibiotic is applied to prevent bacterial growth

13. The method of claim 11, wherein the glutaraldehyde solution is of at least 0.25%

14. The method of claim 11, wherein the glutaraldehyde solution is applied to the target tissue for at least 24 hours.

15. The method of claim 11, wherein the target tissue comprises swine pulmonary visceral pleura.

16. The method of claim 11, wherein the protease inhibitor comprises PMSF.

17. The method of claim 11, wherein the antibiotic is chosen from the group comprising penicillin or streptomycin.

18. The method of claim 11, wherein the antibiotic is a combination of two or more antibiotics.

19. The method of claim 11, wherein the target tissue is first exposed to the at least one hypoosmotic solution, followed by the at least one hyperosmotic solution.

20. The method of claim 11, wherein the target tissue is exposed to the at least one hypoosmotic solution and the at least one hyperosmotic solution, and the antibiotic is applied during the exposure to the at least one hypoosmotic solution and the at least one hyperosmotic solution.
